(19) United States
(12) Patent Application Publication
(10) Pub. No.: US 2016/0262796 A1
(43) Pub. Date: Sep. 15, 2016

(54) Extended Sole and Heel Care Device

(71) Applicants: Richard Craig Coates, Hurlington Beach, CA (US); Julia Lynn Coates, West Palm Beach, FL (US)

(72) Inventors: Richard Craig Coates, Hurlington Beach, CA (US); Julia Lynn Coates, West Palm Beach, FL (US)

(21) Appl. No.: 14/645,375

(22) Filed: Mar. 11, 2015

(51) Int. Cl. A61B 17/54; A61H 11/00

(52) U.S. Cl. CPC A61B 17/54; A61H 11/00

(57) ABSTRACT

An extended sole and heel care device with strengthened extended handles to be held in each hand and pulled back and forth in a see-saw motion across the sole or heel of the foot to be used for cleaning, removing dry or calloused skin, or to apply lotions and oils. Centered between the structured ends is a flexible tool holding area where different skin care tools can be placed. The strengthened extended handles allow the tool holding area to drape behind the rear heel at an angle that allows for ease in abrading or cleaning the heel. An abrasive strip can be placed in tool area for rougher skin treatments like callus removal on the sole of the foot, or a soft absorbent pad can be attached for easy lotion application at the rear of the heel.

### REFERENCE TO RELATED APPLICATIONS

None.

### STATEMENT CONCERNING GOVERNMENT INTEREST

None.

### BACKGROUND OF THE INVENTION

1. Field of the Invention

The present invention relates generally to a health device, and more particularly to strengthened extensions for the controlled movement of a skin care tool to exfoliate the external layer of skin or to abrade a targeted callus growth with the ability to then use the same device to abrade and clean areas such as the back of the heel.

2. Description of the Prior Art

Many people suffer from calluses and heel fissures. The formation of calluses is caused by an accumulation of dead skin cells that harden and thicken over an area of the foot, elbow, knee and hands. Heel Fissures result from severely cracked heels, and usually occur on both heels. These conditions can be alleviated by abrading, but the locations are difficult to reach by many—especially the aging.

To assist in this effort various extension handles have been devised. At their free ends support cleaning brushes, pumice stone, or other abraders, as exemplified in U.S. Pat. No. 4,532,668 to Slonicki; U.S. Pat. No. 5,444,888 to Withey; U.S. Pat. No. 5,560,068 to Blake; U.S. Pat. No. 6,733,595 to Grillo; and many others. While suitable for the purposes intended, each of the foregoing prior examples obtains its usefulness by way of a long handle which needs to be manipulated by its free end to effect precise movements of each of the individual cleaning, cutting and abrading utensils that are cantilevered in various directions from the other end.

Of course, the same aging processes that makes it difficult for us to lean and bend as we advance in years are even more devastating to our wrists and hands, various other floor and seat mounted cleaning and abrading structures therefore followed, exemplified in U.S. Pat. No. 5,685,828 to Dyck; U.S. Pat. No. 6,740,052 to Regner; U.S. Pat. No. 7,266,857 to Mezyed; and others. Once again, while suitable for their purposes, these assistive structures rely on the relative arm, leg and foot agility of the user, an agility that is equally affected by time.

To resolve these manipulative defects recourse was sought in commercially vended flexible water impervious abrading straps that extend between two handles, each fixed to a respective strap end, as exemplified by the exfoliating strap assembly sold under the model designation SKU#23010 by STARCREST OF CALIFORNIA, 19465 Brennan Ave., Perris, Calif. 92599, or the strap mounted callus remover exemplified in U.S. Pat. No. 5,082,009 issued to Cromer, and many others, in which the abrading surface is reciprocated over the selected sole surface of the user's foot. While quite useful in this form for the removal of the callus growth by use of the flexible abrading strap and sanding band, the strokes are not capable of reaching the rear of the heel.

### SUMMARY OF THE INVENTION

Keeping in mind the above problems and restrictions of the prior art, the object of the present invention is to offer the elderly or those with limited flexibility a device that has the effectiveness of a skin care strap with the ability to still clean and abrade the rear of the heel.

Other and further objects shall become apparent upon examination of the appended illustrations and the description that follows.

Briefly, these and other objects are accomplished within the present invention by providing a generally elongated elastomeric strap that extends as a belt or band between a set of extended handles, with a tool section positioned in the central portion of the strap. These handles extend and are strengthened enough to allow the flexible tool section to drape around the back of the heel, allowing the user to abrade in locations they could not without the handles. The center flexible tool section has the ability to receive different cleaning and abrading tools which attach using hook and loop technology, also known as “Velcro”, “Dual Lock” and “touch fasteners”. Other types of attachment could be snaps or buttons, etc. Examples of these cleaning and abrading tools include: an abrasive sheet, brush, loofa, terry cloth material, etc. When each handle of the strap is held, the user can abrade the sole of their foot by holding the handles down (perpendicular to the ground) and pulling back and forth. Or, if the user points the handles forward (parallel to the ground) and lets the flexible strap section drop, while in a seated position with their leg in front of them, they can now position the tool area on the back of the heel and abrade that area by using a back and forth motion.

### BRIEF DESCRIPTION OF THE SEVERAL VIEWS OF THE DRAWINGS

FIG. 1 is an isometric top view of the extended sole and heel care device with an exploded view of a foot care tool and attachment system.

FIG. 2 presents an isometric side view of the extended sole and heel care device with an exploded view of a foot care tool.

FIG. 3 is a side view of the extended sole and heel care device, showing its position during sole abrading.

FIG. 4 is a side view of the extended sole and heel care device, showing its position during heel abrading.

FIGS. 5A-5E is a top view showing several types of body cleaning and abrading tools.

### DESCRIPTION OF THE PREFERRED EMBODIMENT

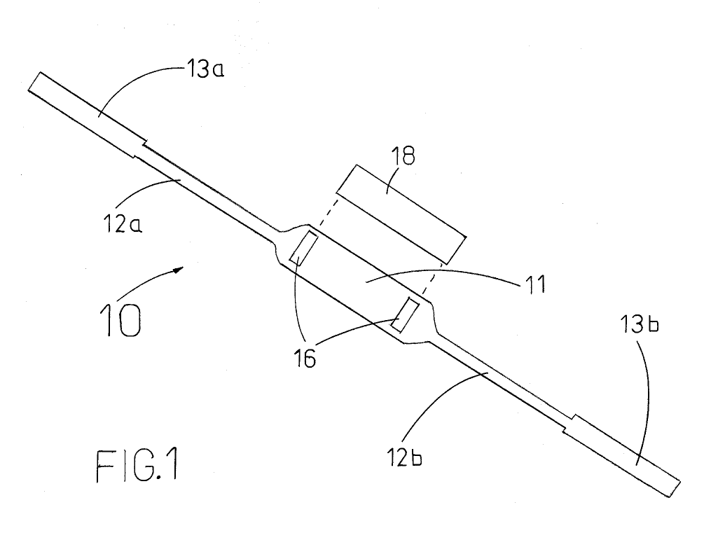

The accompanying drawing FIG. 1 illustrates the extended sole and heel care device of the present invention indicated generally by the numeral 10. Device 10 comprises flexible tool section 11, on each end of tool section 11 flexible strap sections 12a and 12b are connected continuing out from strap section 12a and 12b strengthened handle sections 13a and 13b. Handles 13a and 13b are strengthened enough so when they are held out in front of a user they extend the reach of the user.

Device 10 would be molded from a flexible material such as Silicone, Urethane, or a flexible Poly Vinyl Chloride (PVC). The flexible tool section 11, and flexible strap sections 12a and 12b would be molded thin enough that the material would easily bend. Strengthened handle sections 13a and 13b would be of a more rigid thickness. Of course, device 10 could be molded in pieces and then assembled. If molded separately flexible tool section 11, and flexible strap section 12a and 12b, are molded out of a flexible polymer such as Poly Vinyl Chloride (PVC), Silicone, Urethane, or many others. Strengthened handle sections 13a and 13b would be molded out of stiffer material like Nylon, ABS, or other rigid polymer. The hook side of hook and loop technology 16 is attached to tool section 11, the loop side (not shown) is positioned on the underside of added skin care tool 18. Skin care tool 18 in this drawing is an abrasive.

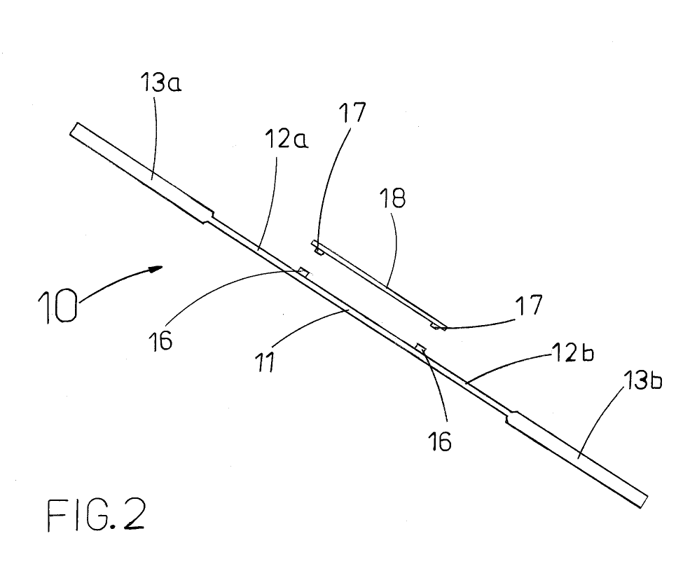

FIG. 2 is a side view of device 10 with the addition of a skin care tool 18 and the hook 16 and loop 17 fasteners, again we have the flexible tool section 11, and the flexible strap sections 12a and 12b along with the strengthened handles 13a and 13b.

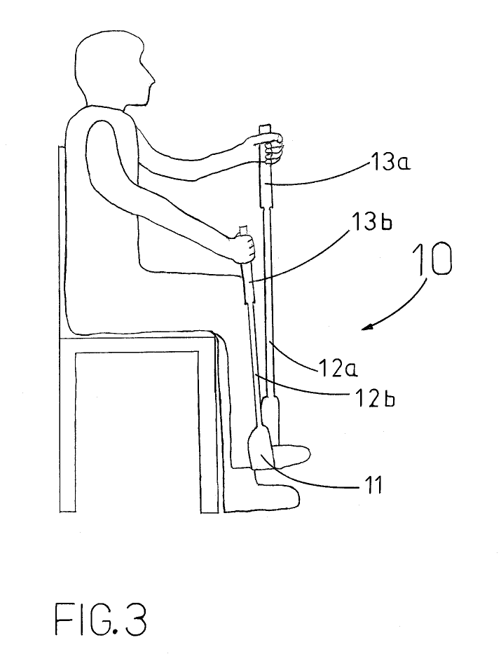

FIG. 3 shows device 10 being used with the user in a seated position, tool section 11 is underneath the users foot and in each of the users hands extended handle 13a and in the other handle 13b. Skin care tool (not shown) can now be pulled back and forth, as handle 13a and flexible strap section 12a goes down strap section 12b and handle 13b is pulled up. The directions are then switched, handle 12a is pulled up and handle 12b then goes down.

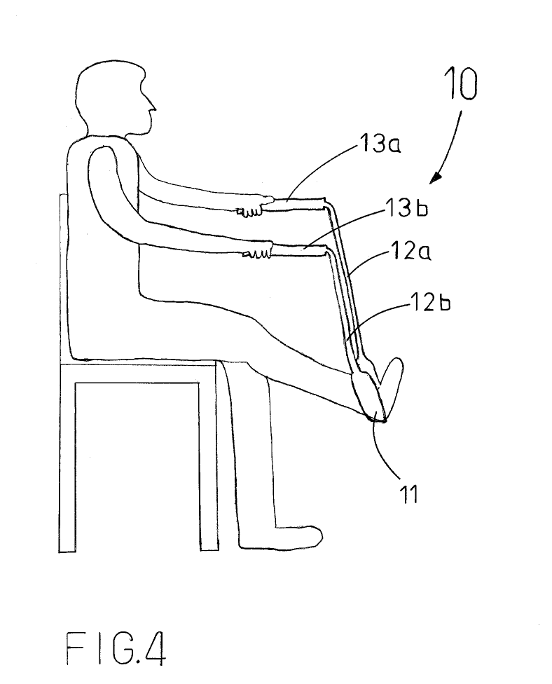

With the user again sitting in FIG. 4 we can see how the strengthened extended handles 13a and 13b extend the reach of the user on device 10 allowing the user to position tool section 11 around the back of the user's heel creating an angle that the user can now clean and abrade the rear of the heel without tool section 11 slipping off of the heel. If strengthened handles 13a and 13b have a slight flex or bend this flex can be compensated for by a longer handles 13a and 13b and device 10 will still create a working angle to clean and abrade the rear of the user's heel.

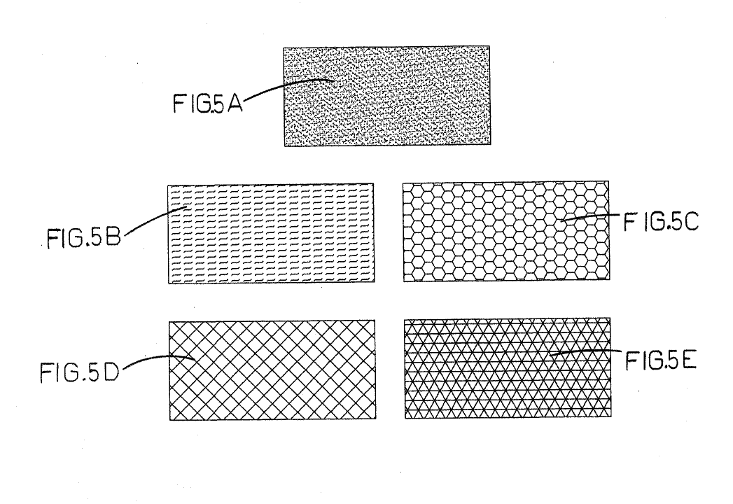

FIG. 5A-5E shows several of the removable body cleaning and abrading tools. FIG. 5A represents an abrasive sheet used for the treatment of calluses. FIG. 5B represents a brush, FIG. 5C designates a loofa pad, for light exfoliating. FIG. 5D portrays a sponge, FIG. 5E represents an absorbent pad that could be filled with a liquid soap, lubricant or medicine.

Obviously many modifications and variations of the instant invention can be effected without departing from the spirit of the teachings herein. It is therefore intended that the scope of the invention be determined solely by the claims appended hereto.

What is claimed is:

1. An extended sole and heel care device comprising:
a structure having two opposing sides, first and second ends;
a flexible tool section located between said first and second ends;
strengthened handles and flexible sections arranged longitudinal to said tool section;
said structure is of ample length for a user sitting generally in an upright position to locate said tool section underneath user's foot; and
said handles are of sufficient length to permit a user to position said tool section on the back part of the foot below the ankle.

2. The extended sole and heel care device as defined in claim 1, wherein said structure comprises a material selected from the group consisting of polyurethane, polyvinyl chloride (PVC), silicone, acrylic polymers, polyvinyl acetate (PVA), and combinations thereof.

3. The extended sole and heel care device as defined in claim 2, wherein said strengthened handles, said flexible sections are molded as a single piece, said material would measure on a Shore D hardness scale

4. The extended sole and heel care device as defined in claim 1, wherein an abrasive sheet is supported to said tool section.

5. The extended sole and heel care device as defined in claim 1, wherein an abrasive screen is supported to said tool section.

6. The extended sole and heel care device as defined in claim 1, wherein a brush is supported to said tool section.

7. The extended sole and heel care device as defined in claim 1, wherein a sponge is supported to said tool section.

8. The extended sole and heel care device as defined in claim 1, wherein a massaging device is supported to said tool section.

9. The extended sole and heel care device body as defined in claim 1, wherein tools are supported to said tool section using hook and loop technology.

10. The extended sole and heel care device as defined in claim 1, wherein abrading material has been embedded into surface of said tool section.

11. The extended sole and heel care device as defined in claim 1, wherein a skin care tool is supported to each opposing side of said tool section.

12. An extended sole and heel care device comprising:
a flexible device with first and second ends and opposing sides;
a flexible tool section located between said first and second ends;
a strengthened handle attached to said first end of said flexible device and second strengthened handle attached to said second end of said flexible device;
said device is of ample length for a user sitting generally in an upright position to locate said tool section underneath user's foot; and
said handles are of sufficient length to permit a user to position said tool section on the back part of the foot below the ankle.

13. The extended sole and heel care device as defined in claim 12, wherein said flexible device comprises a material selected from the group consisting of polyurethane, polyvinyl chloride (PVC), silicone, acrylic polymers, polyvinyl acetate (PVA), and combinations thereof.

14. The extended sole and heel care device as defined in claim 12, wherein an abrasive sheet is supported to said tool section.

15. The extended sole and heel care device as defined in claim 12, wherein an abrasive screen is supported to said tool section.

16. The extended sole and heel care device as defined in claim 12, wherein a sponge is supported to said tool section.

17. The extended sole and heel care device as defined in claim 12, wherein a massaging device is supported to said tool section.

18. The extended sole and heel care device as defined in claim 12, wherein tools are supported to said tool section using hook and loop technology.

19. The extended sole and heel care device as defined in claim 12, wherein a skin care tool is supported to each opposing side of said tool section.
